(19) United States
(12) Patent Application Publication
(10) Pub. No.: US 2016/0212957 A1
(43) Pub. Date: Jul. 28, 2016

(54) Guzmania 'Cardena'

(71) Applicants: Elly Bak, Rijsenhout (NL); Nicolaas Steur, Oude Niedorp (NL)

(72) Inventors: Elly Bak, Rijsenhout (NL); Nicolaas Steur, Oude Niedorp (NL)

(21) Appl. No.: 14/602,903

(22) Filed: Jan. 22, 2015

(51) Int. Cl. A01H 5/02

(52) U.S. Cl. CPC A01H 5/02

(57) ABSTRACT

A new and distinct Guzmania hybrid named ‘CARDENA’ characterized by solid growth habit; funnel-form rosette plant, measuring about 55 cm in height (above the pot when flowering); numerous, green colored foliage (measuring about 50 cm length and about 3-4.5 cm in width). Superior floral bract production; bracts are red-purple in color (closest to RHS 59A), compound inflorescence, measuring about 15 cm in height and about 25 cm in diameter; and long-lasting habit.

### FIELD OF THE INVENTION

The present invention relates to a new, distinct and stable hybrid of Guzmania hybrid, hereinafter referred it as CARDENA′. The present invention relates to seeds which are the Guzmania hybrid ‘CARDENA’, as well as, plants and the plant parts produced by these seeds which have all the morphological and physiological characteristics of the Guzmania hybrid ‘CARDENA’. The present invention also relates to methods for producing these seeds and plants of the Guzmania hybrid ‘CARDENA’. Furthermore, the present invention relates to method of producing progeny Guzmania plants by crossing Guzmania ‘CARDENA’, as either the female or seed or male or pollen parent, with another Guzmania plant and selecting progeny.

### BACKGROUND OF THE INVENTION

The present invention relates to a new, distinct and stable hybrid of Guzmania hybrid, and hereinafter referred to by the variety denomination ‘CARDENA’. The new Guzmania ‘CARDENA’ originated from a cross made in a controlled breeding program by the inventors in 2010, and then first flowered in 2012, in Assendelft, the Netherlands. The female or seed parent is the Guzmania lingulata inbred line identified by code 83275 (unpatented). The male or pollen parent is the Guzmania wittmackii inbred line identified by code 100118122 (unpatented).

Guzmania is a member of the Bromeliaceae family. Guzmania is predominantly epiphytic with a few terrestrial species and is native to the tropics. For the most part, species vary in diameter from 7 or 8 inches to 3 or 4 feet and have rosettes of glossy, smooth-edged leaves.

Floral bracts of Guzmania frequently have brilliant colors and may last for many months. The range of colors for Guzmania is generally from yellow through orange but may also include flame red and red-purple. White or yellow, tubular, three-petalled flowers may also appear on a stem or within the leaf rosette but are usually short-lived.

Guzmania may be advantageously grown as pot plants for greenhouse or home use. Typically, the plants are shaded from direct sunlight. During the spring to autumn period, the central vase-like part of the leaf rosette is normally filled with water.

Guzmania is native to tropical America. Leaves of Guzmania are usually formed as basal rosettes which are stiff and entire and in several vertical ranks Guzmania plants have terminal spikes or panicles which are often bracted with petals united in a tube about as long as the calyx. The ovary is superior and the seeds plumose.

Asexual propagation of Guzmania is frequently performed by vegetative means through the use of tissue culture practices. Propagation of Guzmania can also be from offshoots which can be detached from the mother plant and grown in an appropriate soil or bark mixture.

Methods for cultivation and crossing of Guzmania are well known. For a detailed discussion, reference is made to the following publications, which are incorporated herein by reference: Benzing, David H., THE BIOLOGY OF THE BROMELIADS, Mad River Press, Inc., Eureka (1980); Zimmer, Karl, BROMELIEN, Verlag, Paul Parey, Berlin (1986); and Rauh, Werner, BROLELIEN, Verlag Eugen Ulmer, Stuttgart (1981).

A Guzmania inbred is produced by brother/sister crossing over several generations to produce a genetically homozygous plant selection. A hybrid cultivar is produced by crossing two genetically distinct inbred lines, collecting seeds produced by the cross, and germinating seeds so-produced to make hybrid plants. The hybrid seeds and plants produced by this method are uniform with respect their morphological and physiological characteristics.

A need exists for a greater variety of Guzmania cultivars with attractive ornamental features. Additionally, a need exists for additional Guzmania hybrid cultivars that can be easily propagated by seed. The new Guzmania ‘CARDENA’ was developed through a controlled breeding program and exhibits unique, desirable and stable characteristics.

### SUMMARY OF THE INVENTION

The present invention provides Guzmania plant selections that are solid, medium-sized, long-lasting hybrids with superior bract production and red-purple inflorescence that exhibits good keeping quality. The present invention also provides Guzmania plant selections with a compound inflorescence with a unique red-purple color which distinguishes the new cultivar from typical Guzmania.

These and other objectives have been achieved in accordance with the present invention which provides ‘CARDENA’ as new Guzmania cultivar that is a product of a planned breeding program conducted by the inventors, Elly Bak and Nico D. M. Steur, in Assendelft, The Netherlands, in 2010. The female or seed parent is the Guzmania lingulata inbred line identified by code 83275 (unpatented). The male or pollen parent is the Guzmania witmackii inbred line identified by code 100118122 (unpatented).

Both parental cultivars have a sufficient degree of homozygosity such that the progeny of the cross are genetypically and phenotypically uniform. The new hybrid ‘CARDENA’ therefore can be produced by sexual reproduction by crossing the parental inbred lines identified by the codes 83275 and 100118122 to produce a population of progeny plants, each of which has the combination of characteristics as herein disclosed for the new hybrid ‘CARDENA’.

Seeds which are the hybrid ‘CARDENA’ are produced by crossing the parental inbred lines identified by the codes 83275 and 100118122, and are to be deposited with the American Type Culture Collection, 10801 University Boulevard, Manassas, Va. 20110-2209. ATCC Patent Deposit Designation No. PTA-2500 seeds were deposited with the ATCC on.

### OBJECTS OF THE INVENTION

The present invention related to seeds which produce Guzmania hybrid ‘CARDENA’. The present invention also relates to Guzmania plants, and parts thereof, having all the physiological and morphological characteristics of Guzmania hybrid ‘CARDENA’. The present invention relates to a plant produced from seeds which are Guzmania hybrid ‘CARDENA’. The present invention also relates to plant parts, such as pollen, seeds or inflorescence produced by Guzmania hybrid ‘CARDENA’.

The present invention relates to a method of producing seed which are Guzmania hybrid ‘CARDENA’, by a crossing Guzmania lingulata inbred line identified by code 83275 (unpatented) as the female or seed parent with Guzmania wittmackii inbred line identified by code 100118122 (unpatented) as the male or pollen parent, harvesting seeds produced from said cross.

The present invention also relates to a method of producing plants having all the physiological and morphological characteristics of the Guzmania hybrid ‘CARDENA’ comprising the steps of (a) crossing Guzmania lingulata inbred identified by code 83275 (unpatented) as a female or seed parent with Guzmania wittmackii inbred line identified by code 100118122 (unpatented) as the male or pollen parent. (b) harvesting seeds produced from said cross; and (c) producing plants from said harvested seeds.

The present invention also relates to producing progeny plants from the cross of Guzmania hybrid ‘CARDENA’, as the female or male parent, with another Guzmania plant, and selecting progeny plants from this cross.

### BRIEF DESCRIPTION OF THE PHOTOGRAPH

The patent or application file contains one drawing executed in color. Copies of this patent or patent application publication with color drawings will be provided by the Office upon request and payment of the necessary fees.

The accompanying photograph illustrates the overall appearance of the new Guzmania hybrid ‘CARDENA’ showing the colors as true as is reasonably possible with colored reproductions of this type. Colors in the photograph may differ slightly from the color values cited in the detailed botanical description which accurately describes the color of CARDENA′.

FIG. 1 shows a side view perspective of the primary and top bracts produced by a typical potted, flowering plant of ‘CARDENA’, at 12 months of age from potting size.

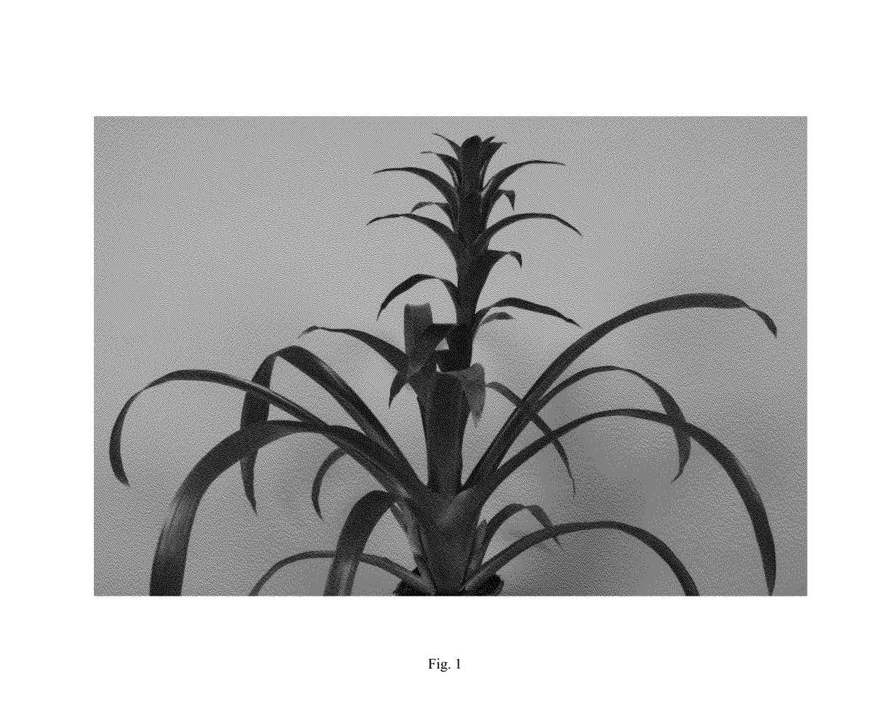

### DETAILED BOTANICAL DESCRIPTION

The present invention was created by the inventors, Elly Bak and Nicolaas D. M. Steur in 2010, and flowered for the first time in 2012 in Assendelft, The Netherlands.

This invention is directed to a Guzmania plant having all the morphological and physiological characteristics of the hybrid ‘CARDENA’ produced from seeds which are the product of crossing the parental Guzmania inbred lines identified by the codes 83275 and 100118122. Both parents have a sufficient degree of homozygosity such that the progeny of the cross were, and continue to be, phenotypically uniform. The new hybrid CARDENA′ can therefore be produced by sexual reproduction by crossing of the inbred selections identified by the codes 83275 and 100118122 to produce a population of progeny plants, each of which has the combination of characteristics herein disclosed for the new hybrid CARDENA′.

The new hybrid ‘CARDENA’ can also be produced by asexually reproducing progeny from the cross of the parental inbred lines identified by the codes 83275 and 100118122. Asexual reproduction of the new cultivar by vegetative means by cuttings was first performed in 2012, in Assendelft, the Netherlands. The first ‘CARDENA’ plants propagated through the use of such cuttings flowered in 2014, in Assendelft, the Netherlands, and have demonstrated that the new cultivar reproduces true-to-type and that the combination of characteristics as herein disclosed for the new cultivar are firmly fixed and retained through successive generations of asexual reproduction.

### BRIEF DESCRIPTION OF THE INVENTION

The following traits have been repeatedly observed and are determined to be unique characteristics of ‘CARDENA’ which in combination distinguish this Guzmania as a new and distinct cultivar:
1. Stemless growth habit;
2. Funnel-form rosette plant, measuring about 55 cm in height (above the pot when flowering);
3. Numerous, green colored foliage (measuring about 50 cm in length and color 3.0 to 4.5 cm in width.
4. Superior floral bract production;
5. Bracts are red-purple in color (closet to RHS 59A)
6. Compound inflorescence, measuring about 15 cm in height, when flowering and about 25 cm in diameter
7. Long-lasting floral bracts.

Of the many commercial cultivars known to the present inventors, the most similar in comparison to the new Guzmania hybrid ‘CARDENA’ is the Guzmania cultivar TUNA′, unpatented. Plants of the new hybrid ‘CARDENA’ differ from plants of ‘LUNA’ primarily in the shape and the color of the inflorescence.

‘CARDENA’ has not been tested and observed under all possible environmental conditions. The phenotype of the new cultivar may vary with variations in environment such as temperature, light intensity, frequency of fertilization, composition of fertilizer, flowering treatment, day length and humidity, without any change in the genotype of the plant.

For example, substantial differences in plant height and diameter; number of leaves, can result depending on the size of the plant at the time that flowering is induced by flowering treatment. Since treatment to include flowering disrupts normal watering and fertilization regiments. Flowering treatment of relatively smaller plants adversely affects the growth of the plant.

The aforementioned photographs, together with the following observations, measurements and values describe the new Guzmania ‘CARDENA’ as grown in a greenhouse in Assendelft, the Netherlands, under conditions which closely approximate those generally used in commercial practice. Plants of ‘CARDENA’ were grown in a greenhouse with day temperatures ranging from 20° C. to 28° C. and night temperatures ranging from 18° C. to 23° C. No artificial lighting or photoperiodic treatments were conducted, but the plants of ‘CARDENA’ are forced into flowering. The following fertilizer is added when growing plants of ‘CARDENA’: 1 part nitrogen, 0.6 parts phosphor, 2 parts Kalium and 0.1 part magnesium.

Color reference are made to the Royal Horticultural Society Colour Chart (RHS), 2001 edition, except where general colors of ordinary significance are used. Color values were taken under daylight conditions in a greenhouse in Assendelft, The Netherlands. The age of the plants of ‘CARDENA’ described is about 17 weeks after flowering treatment.

Classification;
Botanical: Guzamania sp.

Parentage:
Female Parent: Guzmania lingulata inbred line identified by code 83275 (unpatented)
Male parent: Guzmania wittmackii inbred line identified by the code 100118122 (unpatented)

Plant:
General Appearance and Form:
   Height About 55 cm (when flowering)
   Width: About 70 cm
   Shape: Funnel form rosette
Growth habit: Stemless
Plant Vigor: Good
Flowering Season: A fully grown plant can flower year round, starting 16-18 weeks after induction of natural light or through flowering treatment.
Cold Tolerance: Frost tender. Temperatures below 5° C. may damage plants. Fragrance: None

Foliage:
Quantity: About 14 (depending on the size of the plant)
Size of Leaf:
   Length: About 50 cm (when flowering)
   Width: About 3 cm to 4.5 cm
Overall Shape: Lanceolate
Apex Shape: Acuminate
Base Shape: Strap-like around central axis
Margin: Entire
Texture: Smooth
Orientation: Leaf blades arch continuously from base.
Color: Leaf color can vary somewhat depending on the growing conditions.
   Immature and Mature:
      Upper surface: Green, closest to RHS 137A
      Under surface: Green closest to RHS 137B
Venation: None

Inforescence:
Borne: Erect
Shape: Compound
Size:
   Length: About 15 cm in height when flowering
   Diameter: About 25 cm
Time of Bloom: A fully grown plant can produce an inflorescence containing about 120 flowers (depending on the size of the plants), and can bloom the whole year starting 16-18 weeks after natural induction or through flowering treatment. Duration of Bloom: Each flower blooms one (1) day and the total blooming of the whole inflorescence is about 6 weeks.

### Petal:

Number: 3 per flower
Length: About 6 cm
Width: About 0.5 cm

### Overall Shape: Ligulate

### Apex Shape: Obtuse

### Base Shape: Fused

### Color:

Upper and under surfaces: White, closest to RHS 155B

### Sepals:

Number: 3 per flower
Length: About 3.5 cm
Width: About 0.3 cm
Overall Shape: Ligulate
Apex Shape: Acute
Base Shape: Fused
Color:
   Upper and under surfaces: White, closest to RHS 155B

Bracts:
Scape Bracts:
   Quantity: About 10
   Arrangement: Alternate
   Size:
      Length: About 40 cm (lowest) to 18 cm (scape bracts positioned just below the primary bracts).
      Width: About 3.5 to 4 cm
      Overall shape: Linear lanceolate
      Apex shape: Acute
      Base shape: Fused
      Margin: Entire
      Texture: Smooth
      Color:
         Upper and under surfaces:
      Scape bracts are green, closest to RHS 137A, with red-purple closest to RHS 59A.
      Primary Bracts:
         Quantity: About 12
      Arrangement: Alternate
      Size:
         Length: About 18 cm (lowest) to about 8 cm (primary bracts become shorter closer to the top of the plants).
         Width: About 2.5 cm to 3 cm
      Overall shape: Recurved and ovate-lanceolate
      Apex shape: Acute
      Base shape: Fused
      Margin: Entire
      Texture: Smooth
      Color:
         Upper and under surfaces: Red-purple, closest to RHS 59A.
      Floral bracts: Disposed within the inflorescence.

Reproductive Organs:
Androecium:
Stamen:
   Number: 6 per flower
   Length: About 5 cm
   Diameter: About 1 mm
   Color: White, too small to distinguish RHS value.
Anther:
   Length: About 0.5 cm
   Color: Cream, too small to distinguish RHS value.
Pollen:
   Amount: None

### Gynoecium:

Pistil:
   Number: 1 per flower
   Length: About 5.5 cm
Stigma:
   Shape: 3-parted
   Width: About 0.3 cm
   Color: White, too small to distinguish RHS value
Style:
   Length: About 4.5 cm
   Color: White, too small to distinguish RHS value.
Ovary:
   Position: Superior
   Shape: Conical
   Length: About 0.7 cm
   Diameter: About 0.3 cm
   Color: Light Green, closest to RHS 157A

SEED/FRUIT: Sterile hybrid, no seed or fruit produced

DISEASE/PEST RESISTANCE: No observations made

DISEASE/PEST SUSCEPTIBILTY: No observations made

We claim:

1. A Guzmania plant named ‘CARDENA’, representative seed to be deposited at the American Type Culture Collection (ATCC). with Patent Deposit Designation No.: PTA-

2. Guzmania seed to be deposited With the American Type Culture Collection.

3. Plant parts obtained from Guzmania plant of claim 1.

4. (canceled)

5. (canceled)
